(19) United States
(12) Patent Application Publication
(10) Pub. No.: US 2018/0085382 A1
(43) Pub. Date: Mar. 29, 2018

(54) COMPOSITIONS AND METHODS FOR COGNITIVE HEALTH AND MEMORY

(71) Applicant: Script Essentials, LLC, Broomfield, CO (US)

(72) Inventor: Suzy Cohen, Broomfield, CO (US)

(21) Appl. No.: 15/707,966

(22) Filed: Sep. 18, 2017

Related U.S. Application Data
(60) Provisional application No. 62/401,048, filed on Sep. 28, 2016.

(51) Int. Cl. A61K 31/593; A61K 35/60; A61K 31/202; A61K 31/20; A61K 9/00

(52) U.S. Cl. CPC A61K 31/593; A61K 35/60; A61K 9/0053; A61K 31/20; A61K 31/202

(57) ABSTRACT

The present disclosure is a compound, a method of making the compound and method of using such compound preferably in the form of a dietary supplement that, when administered, is capable of treating heart disease and heart-related disorders, among other symptoms. The unique combination of the composition is preferably administered orally. The composition is preferably comprised of about 1,000 to 5,000 IU of vitamin D, preferably cholecalciferol, about 250 to 1,250 mg of fish oil concentrate, about 200 to 1,200 mg of omega-3 fatty acids, about 200 to 700 mg of DHA, about 20 to 125 mg of EPA, and about 10 to 150 mg of other fatty acids. The composition can further comprise a palliative agent, and can be provided in the form of a softgel, powder, liquid or tablet.

### CROSS-REFERENCE TO RELATED APPLICATIONS

This application claims priority under 35 U.S.C. §119(e) to U.S. Provisional Application Ser. No. 62/401,048, filed Sep. 28, 2016, which is incorporated herein in its entirety by reference.

### FIELD OF THE INVENTION

The present disclosure is directed to compounds, particularly dietary supplements, and methods for formulating and administering the same. More particularly, the present disclosure relates to compositions and methods for treating heart disease and other symptoms, preferably comprising vitamin D, fish oil concentrate, omega-3 fatty acids, DHA, EPA and/or other fatty acids.

### BACKGROUND OF THE INVENTION

Vitamin D is essential to the healthy growth, development and healthy function of bones, teeth, and muscles. Studies have shown that vitamin D intake also promotes healthy heart function and strengthens the human immune system. However, most individuals do not consume the recommended daily amounts of vitamin D necessary to maintain healthy function of these vital organs and tissue. Furthermore, many individual diets do not include essential fatty acids, such as Eicosapentaenoic Acid (EPA) and Docosahexaenoic Acid (DHA), which may reduce the risk of coronary heart disease. For example, most people do not consume necessary levels of important omega-3 fatty acids. Deficiencies in these important vitamins and fatty acids can lead to problems with circulation and arterial function, which in turn may lead to higher cholesterol and increase the risk of stroke or heart disease. And finally, the American Pregnancy Association supports the use of DHA before and during pregnancy, for both the baby and the mother.

It is therefore desirable to provide a dietary supplement that increases absorption of Eicosapentaenoic Acid (EPA) and Docosahexaenoic Acid (DHA), and that addresses the problems associated with heart disease, including but not limited to cardiovascular disorders, coronary heart disease, fatal ischemic events (i.e., stroke and heart attacks), and that otherwise prevents inflammation while promoting healthy cholesterol levels. In another aspect of the present disclosure, a compound is provided in the form of a dietary supplement that promotes healthy brain development and/or function. These and other problems are addressed by the compositions and methods described in detail below.

### SUMMARY OF THE INVENTION

In varying embodiments described herein, the invention relates to a compound, particularly in the form of a dietary supplement, which improves upon the healthy function of the heart, arteries and circulatory systems in the human body. The invention further comprises compositions that prevent inflammation, promote healthy cholesterol levels, and reduce the risk of heart disease.

In varying embodiments described herein, the invention relates to a composition that is capable of treating the problems described above and elsewhere in this disclosure, and specifically heart disease and cardiovascular-related disorders. The composition in preferred embodiments is preferably comprised of a unique and novel formulation in predetermined amounts, and further provides benefits previously unexpected. Certain elements of the novel compounds and methods for formulating the same are described in varying levels of detail herein.

In one embodiment, the composition comprises a prescribed amount of DHA or combination of DHA and EPA. Independent investigation has shown that individuals with low blood levels of DHA or DHA and EPA (combined) face significantly greater risk for coronary heart disease, fatal ischemic events such as stroke, or even sudden death. Fish oils, a source of omega-3 fatty acids, may include both DHA and EPA fats, and have generally been shown to prevent heart disease. In addition, omega-3 fatty acids (including those from fish oils) may help maintain healthy cholesterol levels. Independent study has also shown that fish oils reduce triglycerides in your blood.

Investigation has determined that omega-3 fatty acids, and DHA and EPA in particular, protect healthy heart and cardiovascular functioning in several ways. These fatty acids interfere with the formation of dangerous clots and inhibit the production and release of pain-causing inflammatory compounds, which in turn cause plaque formation in the arteries. Therefore, according to a preferred embodiment, the composition comprises fatty acids included with one or more fish oils.

According to another embodiment, the composition comprises an amount of DHA sufficient to promote healthy brain development and function. Research has shown that the cerebral cortex is composed of 20% fatty acids, and DHA in particular may be found in a patient's neurons (cells located in the brain and spinal cord). A quantity of DHA is therefore provided with the composition to support the structural integrity of the human brain and central nervous system. DHA, in varying levels described herein, may further be administered in the form of a supplement or otherwise to promote healthy mood and emotional well-being, as well as to improve cognitive function. By way of example but not limitation, a prescribed amount of

DHA may be administered to maintain cellular fluidity and improve memory and/or thinking patterns. In one embodiment, the composition comprises a prescribed amount of DHA to treat patients with a traumatic brain injury.

DHA may also be administered during pregnancy to promote healthy nervous system function in developing babies, or to improve immunity and mood support for the expecting mother throughout pregnancy. Accordingly, one embodiment of the present invention comprises a supplement having a prescribed amount of DHA to accomplish these objectives as well.

In one embodiment, the composition comprises a combination of DHA or other fatty acids with vitamin D. This combination in particular may address mental decline, reduce risk for Alzheimer's disease or dementia, and in general improve cognitive functioning among older individuals.

According to one particular aspect, a compound is provided that comprises about 1,000 to about 5,000 IU of vitamin D, about 250 mg to about 1,250 mg of fish oil concentrate, about 20 mg to about 125 mg of eicosapentaenoic acid, about 200 mg to about 700 mg of docosahexaenoic acid and about 10 mg to about 150 mg of other omega-3 fatty acids.

According to yet another aspect, a compound is provided that comprises about 2,500 IU of vitamin D, about 750 mg of fish oil concentrate, about 570 mg of omega-3 fatty acids and about 70 mg of other fatty acids.

Other nutrients and compounds may be supplied with the compositions described herein to provide further benefits to the heart, cardiovascular system, nervous system and/or brain, and are described in greater detail below.

In some embodiments, the composition can be provided as a dietary supplement in the form of a capsule that is coated to withstand stomach acid. In some embodiments, the composition can be administered in the form of an acid-resistant or enteric-coated tablet. In yet other embodiments, the composition can be provided as a powder or in a liquid form. Methods for formulating the compound are also disclosed herein. In one aspect of the present disclosure, a method is provided that comprises the steps of administering an effective amount of a composition to a patient suffering from heart disease, wherein the composition comprises about 1,000 to 5,000 IU of vitamin D, about 250 to 1,250 mg of fish oil concentrate, about 200 to 1,200 mg of omega-3 fatty acids, about 200 to 700 mg of DHA, about 20 to 125 mg of EPA, and about 10 to 150 mg of other fatty acids.

The unique combination of the composition is preferably administered orally in the form of a softgel. The unique combination has synergistic advantages over previously known compositions. As disclosed in more detail in the Detailed Description, the present invention provides compositions, methods for treating heart disease and heart-related disorders, and methods for forming the compound.

These and other advantages will be apparent from the disclosure of the invention(s) contained herein. The above-described embodiments, objectives, and configurations are neither complete nor exhaustive. The Summary of the Invention is neither intended nor should it be construed as representative of the full extent and scope of the present invention. References made herein to “the present invention” or aspects thereof should be understood to mean certain embodiments of the present invention and should not necessarily be construed as limiting all embodiments to a particular description. The present invention is set forth in various levels of detail in the Summary of the Invention and the Detailed Description, and no limitation as to the scope of the present invention is intended by either the inclusion or non-inclusion of elements in this Summary of the Invention. Additional aspects of the present invention will become more readily apparent from the Detailed Description.

### DETAILED DESCRIPTION

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

As used herein, the phrases “at least one”, “one or more”, and “and/or”, as used herein, are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Unless otherwise indicated, all numbers expressing quantities, dimensions, conditions, and so forth used in the specification and claims are to be understood as being modified in all instances by the term “about”.

The term “substantially free of” as used herein should be understood to mean the specified items make up less than 10% of the composition.

The term “a” or “an” entity, as used herein, refers to one or more of that entity. As such, the terms “a” (or “an”), “one or more” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

It shall be understood that the term “means” as used herein shall be given its broadest possible interpretation in accordance with 35 U.S.C. Section 112(f). Accordingly, a claim incorporating the term “means” shall cover all structures, materials, or acts set forth herein, and all of the equivalents thereof. Further, the structures, materials, or acts and the equivalents thereof shall include all those described in the summary of the invention, brief description of the drawings, detailed description, abstract, and claims themselves.

### Composition:

An aspect of the present invention is a composition, preferably in the form of a dietary supplement. The components of the composition listed below can be used in any sub-combination. Thus, the sub-combinations of components are embodiments of the invention.

The composition is preferably comprised of a unique and novel formulation in predetermined amounts, and further provides benefits previously unexpected. In a preferred embodiment, the composition is comprised of at least: vitamin D (as cholecalciferol); fish oil concentrate; omega-3 fatty acids, including eicosapentaenoic acid and/or docosahexaenoic acid; and other omega-3 fatty acids. In certain embodiments, the composition further comprises one or more palatability agents. In other embodiments, the composition is substantially free of gluten, wheat, egg, peanuts, tree nuts, dairy, sugar, corn, soy, fish and shellfish.

According to one embodiment, the composition may further comprise a blend of gelatin softgel (including but not limited to fish gelatin, glycerin, purified water), natural mixed tocopherols, fish oil (including but not limited to anchovy, sardine, mackerel) and fish gelatin (including but not limited to tilapia and carp). Certain embodiments may comprise combinations and/or sub-combinations of the foregoing. These ingredients can be included to promote healthy heart and cardiovascular system function, as well as healthy brain and cognitive function within the human body, and as described in detail herein. In varying embodiments, the composition is preferably administered orally in the form of a capsule, liquid, powder or tablet.

More particularly, a preferred embodiment of the present disclosure comprises the following, with variability in dosages listed below:

Vitamin D (as cholecalciferol)—about 2,500 IU;

Fish oil concentrate—about 750 mg;

Omega-3 fatty acids—about 640 mg;

EPA (eicosapentaenoic acid)—about 70 mg;

DHA (docosahexaenoic acid)—about 500 mg; and

Other omega-3 fatty acids—about 70 mg;

Each of the foregoing are described in greater detail herein.

Vitamin D

In a preferred embodiment, the composition is comprised of vitamin D. In a most preferred embodiment, the compound is comprised of a predetermined amount of the activated form of vitamin D known as “D3” or “Cholecalciferol.” It has been found from experimentation that this form of vitamin D is optimal for human absorption. As autoimmune antibodies are known to attack and damage the endocrine glands of certain individuals, it is desirable to include a predetermined amount of D3 with the composition, which can reduce autoimmune attack. In addition, certain individuals have a deficiency in this fat-soluble vitamin D, and therefore have trouble regulating blood glucose and controlling their immune response. Investigation has shown a strong correlation between vitamin D deficiency and autoimmune diseases. One particular study reveals that individuals who lacked vitamin D had the highest autoantibody levels against the thyroid gland, for example. A deficiency of vitamin D can also cause DNA damage, blunt the immune response, exasperate cardiovascular problems, and cause increased permeability within the intestinal tract, thus intensifying the immune response.

In a preferred embodiment, the composition comprises from about 1,000 to 5,000 IU of vitamin D, preferably cholecalciferol. In a most preferred embodiment, the composition comprises about 2,500 IU of cholecalciferol. In alternate embodiments, one or more additional fat-soluble vitamins are included or substituted for cholecalciferol.

Fish Oil

As described in general above, omega-3 fatty acids may come in different forms. One such form is a “triglyceride” form, and another if a “ethyl ester” form. Triglycerides are made of fatty acids (such as EPA and DHA) and are attached to a glycerol “backbone”. This is the molecular form of fatty acids that makes up virtually all fats and oils. Omega-3 fatty acids that occur in fish and fish oil are almost exclusively triglycerides in form. Ethyl esters, by contrast, are fatty acids that attach to an alcohol molecule, specifically “ethanol.” These ethyl esters are synthetic, and are not naturally occurring in fish.

In a preferred embodiment, the composition comprises from about 250 to 1,250 mg of fish oil concentrate. In a most preferred embodiment, the composition comprises about 750 mg of fish oil concentrate.

Omega-3 Fatty Acids

In a preferred embodiment, the composition comprises omega-3 fatty acids, namely DHA and/or EPA, in a “natural” form as found in a “triglyceride” type of fish oil, and is otherwise not synthetic or refined. In a preferred embodiment, the omega-3 fatty acids contain both EPA and DHA, although preferably a higher percentage of DHA to support brain, heart and behavioral wellness. DHA has several benefits as described above, and has been shown through experimentation to serve as an immune modulator. DHA also has improved efficacy when combined with vitamin D.

In a preferred embodiment, the composition comprises from about 200 to 1,200 mg of omega-3 fatty acids, preferably DHA and EPA. In a preferred embodiment, the composition comprises from about 200 to 700 mg of DHA and about 20 to 125 mg of EPA. In a most preferred embodiment, the composition comprises about 640 mg of omega-3 fatty acids, comprising about 500 mg of DHA and about 70 mg of EPA.

Additional Elements

In a preferred embodiment, the composition comprises from about 10 mg to 150 mg of other fatty acids. In a most preferred embodiment, the composition comprises about 70 mg of other fatty acids. According to one embodiment, the composition may also comprise a blend of gelatin softgel (including but not limited to fish gelatin, glycerin, purified water), natural mixed tocopherols, fish oil (including but not limited to anchovy, sardine, mackerel) and fish gelatin (including but not limited to tilapia and carp). Certain embodiments may comprise combinations and/or sub-combinations of the foregoing.

Other Components

In varying embodiments, the composition can be substantially free of gluten, wheat, egg, peanuts, tree nuts, dairy, sugar, corn, soy, artificial colors, preservatives, fish and shellfish, and combinations thereof.

Palatability

The composition is preferably provided in a softgel form. In alternate embodiments, the composition is administered in the form of a gummy chew, tablet, powder or liquid extract. In varying embodiments, the composition can further comprise variances in regards to encapsulation, or powder formulations. According to certain embodiments, the compositions described herein can further be provided with one or more palatability agents. These palatability agents serve to add flavor to the composition so that an effective dosage is more easily ingested. Particularly suitable palatability agents for use in the composition of the present invention include, but are not limited to, plant oils, plant hydrolysates, yeast, yeast hydrolysates, and combinations thereof.

Methods

An aspect of the invention is a method to treat heart disease and other symptoms described herein with a composition comprising vitamin D, fish oil concentrate, omega-3 fatty acids, DHA, EPA and other fatty acids. The patient is treated by providing an effective amount of the composition. By way of example, between 1 and 3 softgels of the composition can be provided to a patient per day, in some embodiments 2 softgels per day. The softgels can be taken by the patient daily, preferably in the morning before any other food is ingested by the patient.

An aspect of the invention is a method to prepare a compound to treat heart disease or other disorders described herein. The method comprises providing proportional amounts of each material such that the resulting composition contains from about 1,000 to 5,000 IU of vitamin D, preferably cholecalciferol, from about 250 to 1,250 mg of fish oil concentrate, from about 200 to 1,200 mg of omega-3 fatty acids, from about 200 to 700 mg of DHA, from about 20 to 125 mg of EPA, and from about 10 to 150 mg of other fatty acids. The components are mixed, then can be provided to a delivery device (for example, softgel).

In one embodiment, the composition is made and/or administered as a dietary supplement. In one embodiment, the composition is administered in the form of a softgel. In an alternate embodiment, the composition is administered in the form of a gummy chew, tablet, powder or liquid extract. In further embodiments, the composition comprises one or more palatability agents to favorably alter the taste of the composition for human consumption.

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims.

Ranges have been discussed and used within the forgoing description. One skilled in the art would understand that any sub-range within the stated range would be suitable, as would any number within the broad range, without deviating from the invention.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

What is claimed is:

1. A composition for improving cognitive health comprising:
about 1,000 to about 5,000 IU of vitamin D;
about 250 mg to about 1,250 mg of fish oil concentrate;
about 20 mg to about 125 mg of eicosapentaenoic acid;
about 200 mg to about 700 mg of docosahexaenoic acid; and
about 10 mg to about 150 mg of other omega-3 fatty acids.

2. The composition of claim 1 comprising about 70 mg of eicosapentaenoic acid.

3. The composition of claim 2 comprising about 500 mg of docosahexaenoic acid.

4. The composition of claim 3 comprising about 70 mg of other omega-3 fatty acids.

5. The composition of claim 4 comprising about 750 mg of fish oil concentrate.

6. The composition of claim 5 wherein the vitamin D is in the form of cholecalciferol.

7. The composition of claim 6 comprising about 2500 IU of cholecalciferol.

8. The composition of claim 7 further comprising at least one palatability agent.

9. The composition of claim 8 wherein the composition is administered orally in the form of a capsule, liquid, powder, chewable or tablet.

10. A composition for improving memory, comprising:
about 2500 IU of vitamin D;
about 750 mg of fish oil concentrate;
about 570 mg of omega-3 fatty acids; and
about 70 mg of other fatty acids.

11. The composition of claim 10 wherein the vitamin D is in the form of cholecalciferol.

12. The composition of claim 10 wherein the omega-3 fatty acids are comprised of eicosapentaenoic acid and docosahexaenoic acid.

13. The composition of claim 12 comprising about 500 mg of docosahexaenoic acid.

14. The composition of claim 13 comprising about 70 mg of eicosapentaenoic acid.

15. The composition of claim 14 further comprising a palatability agent.

16. The composition of claim 15 wherein the palatability agent is selected from the group consisting of plant oils, plant hydrolysates, yeasts and yeast hydrolysates.

17. The composition of claim 16 wherein the composition is administered orally in the form of a capsule, liquid, powder, chewable or tablet.

18. The composition of claim 17 wherein the composition is substantially free of gluten, wheat, egg, peanuts, tree nuts, dairy, sugar, corn, soy, fish and shellfish.

19. A method for treating cognitive health and memory comprising:
administering an effective amount of a composition to a patient suffering from the heart-related disease, wherein the composition comprises:
about 1,000 to 5,000 IU of vitamin D;
about 250 to 1,250 mg of fish oil concentrate;
about 200 to 1,200 mg of omega-3 fatty acids;
about 200 to 700 mg of DHA;
about 20 to 125 mg of EPA; and
about 10 to 150 mg of other fatty acids.

20. The method of claim 19 wherein the vitamin D is in the form of cholecalciferol.
